(19) United States
(12) Patent Application Publication
(10) Pub. No.: US 2021/0019296 A1
(43) Pub. Date: Jan. 21, 2021

(54) SYSTEM AND METHOD FOR DATA DE-DUPLICATION AND AUGMENTATION

(71) Applicant: Surescripts, LLC, Arlington, VA (US)

(72) Inventors: Rachel Lyn Petersen, Inver Grove Heights, MN (US); Gregory Martin Anderson, Washington, DC (US); Ryan Douglas Hess, McLean, VA (US); Christina Marie Janacek, Blaine, MN (US); Christopher Carlo DiBlasi, Purchase, NY (US); Keith Edward Willard, Saint Paul, MN (US); Joseph Jerome Athman, Minneapolis, MN (US); Stacy Ward-Charlerie, Pembroke Pines, FL (US); Timothy James Vincent, Portland, OR (US); Emma Anne Elizabeth Sorteberg, Anoka, MN (US); Kenneth Dale Foster, Golden Valley, MN (US)

(21) Appl. No.: 16/932,525

(22) Filed: Jul. 17, 2020

Related U.S. Application Data
(60) Provisional application No. 62/876,407, filed on Jul. 19, 2019.

(51) Int. Cl. G06F 16/215; G06F 16/22; G06F 16/23; G06F 16/906; G16H 10/60; G16H 20/10; G06N 3/04

(52) U.S. Cl. CPC G06F 16/215; G06F 16/2282; G06F 16/2358; G06N 3/04; G16H 10/60; G16H 20/10; G06F 16/906

(57) ABSTRACT

Methods for data de-duplication and data augmentation are performed by systems and devices. A request for information is received over a network from a requestor by a host, and is provided to an information source for a response. The host retrieves first data from a data source associated with the request, and receives the response that includes second data associated with the request. The first and second data are aggregated by the host. The aggregated data is processed by the host to remove duplicate information/records. The aggregated data is processed by the host to augment eligible data fields to correct, supplement, and calculate the aggregated data through data augmentation. An updated response is then provided back to the requestor with augmented data tracking. Neural network models are also utilized by the host for data augmentation.

### CROSS-REFERENCE TO RELATED APPLICATION(S)

The present application claims priority to U.S. Provisional Patent Application No. 62/876,407, entitled “SYSTEM AND METHOD FOR DATA DE-DUPLICATION AND AUGMENTATION,” and filed on Jul. 19, 2019, the entirety of which is incorporated by reference herein.

### BACKGROUND

Medication history information may be requested by medical care providers in order to determine medications prescribed and/or used by a patient. Various entities may be a party to transactions involving medications, and may provide different portions of data related to such transactions. Additionally, records of transactions may accumulate duplicated data, may lack data for certain fields, and/or may have incomplete or ambiguous data.

### BRIEF SUMMARY

Methods, systems, and apparatuses are described for data de-duplication and data augmentation, substantially as shown and/or described herein in connection with at least one of the figures, as set forth more completely in the claims. Methods for data de-duplication and data augmentation are performed by systems and devices. A request for information is received over a network from a requestor by a host, and is provided to an information source for a response. The host retrieves first data from a data source associated with the request, and receives the response that includes second data associated with the request. The first and second data are aggregated by the host. The aggregated data is processed by the host to remove duplicate information/records. The aggregated data is processed by the host to augment eligible data fields to correct, supplement, and calculate the aggregated data through data augmentation. An updated response is then provided back to the requestor with augmented data tracking. Neural network models are also utilized by the host for data augmentation.

### BRIEF DESCRIPTION OF THE DRAWINGS/FIGURES

The accompanying drawings, which are incorporated herein and form a part of the specification, illustrate embodiments and, together with the description, further serve to explain the principles of the embodiments and to enable a person skilled in the pertinent art to make and use the embodiments.

FIG. 1 shows a block diagram of a computer system that includes a data de-duplicator and a data augmenter, according to an example embodiment.

FIG. 2 shows a block diagram of a network of computer systems that includes the data de-duplicator and the data augmenter of FIG. 1, according to an example embodiment.

FIG. 3 shows a block diagram of a host system for data de-duplication and data augmentation, according to an example embodiment.

FIG. 4 shows a flowchart for data augmentation, according to an example embodiment.

FIG. 5 shows a flowchart for data de-duplication, according to an example embodiment.

FIG. 6 shows a flow diagram for data augmentation utilizing a neural network model, according to an example embodiment.

FIG. 7 shows a graphical representation of a code classification generated by a neural network model, according to an example embodiment.

FIG. 8 shows a graphical representation of a code classification generated by a neural network model, according to an example embodiment.

FIG. 9 shows a block diagram of a processing device/system in which the techniques disclosed herein may be performed and the embodiments herein may be utilized.

Embodiments will now be described with reference to the accompanying drawings. In the drawings, like reference numbers indicate identical or functionally similar elements. Additionally, the left-most digit(s) of a reference number identifies the drawing in which the reference number first appears.

### DETAILED DESCRIPTION

### I. Introduction

The present specification discloses numerous example embodiments. The scope of the present patent application is not limited to the disclosed embodiments, but also encompasses combinations of the disclosed embodiments, as well as modifications to the disclosed embodiments.

References in the specification to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

Furthermore, it should be understood that spatial descriptions (e.g., “above,” “below,” “up,” “left,” “right,” “down,” “top,” “bottom,” “vertical,” “horizontal,” etc.) used herein are for purposes of illustration only, and that practical implementations of the structures described herein can be spatially arranged in any orientation or manner.

Dates referred to herein are provided in the form of Month/Day/Year, (MM/DD/YY), or (MM/DD/YYYY).

Still further, it should be noted that the drawings/figures are not drawn to scale unless otherwise noted herein.

Numerous exemplary embodiments are now described. Any section/subsection headings provided herein are not intended to be limiting. Embodiments are described throughout this document, and any type of embodiment may be included under any section/subsection. Furthermore, it is contemplated that the disclosed embodiments may be combined with each other in any manner. That is, the embodiments described herein are not mutually exclusive of each other and may be practiced and/or implemented alone, or in any combination.

### II. Example Embodiments

The example techniques and embodiments described herein may be adapted to various types of systems and devices, for example but without limitation, computing systems (e.g., computers/computing devices such as desktops, laptops, etc., and servers, enterprise computing systems, etc.), communication devices (e.g., cellular and smart phones, etc.), and/or the like, that communicate information, such as medication information, in different ways, e.g., in accordance with communication standards. For instance, computing systems that communicate over a network and exchange clinical information in accordance with the CCDA standard, or the like, may be configured according to the described embodiments and techniques.

While the embodiments herein may be described with respect to various computing systems and implementations as conceptual and/or illustrative examples for descriptive consistency, other types of electronic and communication devices and implementations are also contemplated for implementing the disclosed techniques. It is contemplated herein that in various embodiments and with respect to the illustrated figures of this disclosure, one or more components described and/or shown may not be included and that additional components may be included.

### A. Example Data De-Duplication Embodiments

In embodiments, data de-duplication is performed. Improving Medication History data thru removing duplicate dispensed medication records prior to sending a response to a requestor/requesting entity/provider vendor, e.g., a doctor, doctor office staff member, other medical care provider, etc., is contemplated according to such embodiments. In some embodiments, data de-duplication may be performed prior to the performance of data augmentation, as described herein. De-duplication may be performed for medical history records, Medication History for Reconciliation (MHR), Medication History for Ambulatory (MHA), Medication History for Long Term Post Acute Care (MH-LTPAC) transactions, and/or the like.

In an example, a method for data de-duplication may be performed by a system or device configured to perform one or more operations thereof. For instance, a requesting provider vendor may send a request for a prescription history (“RxHistoryRequest”) for an eligible patient, and the host system validates the message, passes it to the appropriate pharmacy benefit manager (PBM)/payer and/or the appropriate state Prescription Drug Monitoring Program (PDMP), and looks up pharmacy fill data in the pharmacy database. The host system may validate the level of consent as Yes or No, and check for the date range of the history request. If populated with a Start and End date, a number of medications may be returned to the requestor, e.g., starting with the End date in embodiments. A PBM/payer may then process the RxHistoryRequest and check for the date range of the history request. The PBM/payer may then validate the level of consent is Yes, and if populated with a Start and End date, a number of medications may be returned to the host system, e.g., starting with the end date. The PBM/payer may create a response for the prescription history request (“RxHistoryResponse”) and submit it back to the host that may then validate the RxHistoryResponse. Similarly, a PDMP may also process the RxHistoryRequest and provide an appropriate RxHistoryResponse. The host system may then aggregate Pharmacy Fill data, PDMP data, and/or PBM paid claim data, and generate and send the RxHistoryResponse to the originating requestor.

In embodiments, validating the RxHistoryResponse may include one or more of identifying data elements that are eligible for de-duplication processing, and executing de-duplication or “de-duping” logic on PBM/Payer Claim response data and PDMP data, and removing any duplicated dispensed medication records. In embodiments, aggregating Pharmacy Fill, PDMP, and PBM paid claim data includes at least one of identifying data elements that are eligible for de-duplication processing, and executing de-duplication logic on Pharmacy Fill data, PDMP data, and PBM/Payer claim data (or any combination thereof) and removing any duplicated dispensed medication.

In data de-duplication embodiments, the system may be configured to remove Medication History records from the RxHistoryResponse when duplicate medication history reconciliation medication dispensed records are identified in the PBM paid claim data, PDMP data, and Pharmacy prescription fill data.

The system may be configured to match data when duplicate data is identified in: PBM Paid Prescription Claims compared to other PBM Paid Prescription Claims and/or PDMP data, and/or PBM Paid Prescription claims compared to Pharmacy Prescription Fill Data, in embodiments. When medication records are removed, pharmacy fill or PDMP data may be preferred over paid prescription claim data. The system may be configured to only provide de-duplicated data to Medication History for Reconciliation entities, in some embodiments, and the system may be configured to provide de-duplicated data to any eligible party that has opted in, in other embodiments.

If the MHR customer is also a Medication History for Ambulatory customer, then those customers may also receive de-duplicated data. The system may be configured to provide the ability for Medication History Reconciliation service subscribers to not receive or “Opt Out” from receiving de-duplicated data.

The system may be configured to provide the ability to only allow approved/subscribing customer access to the de-duplicated Medical History data, in embodiments. The system may be configured to provide the ability to “Opt in” and/or “Opt Out” to this de-duplicated Medical History data. This “Opt In”/“Opt Out” functionality may be provided at the sub account (portal) level and at the Health care provider system level (e.g., down to health system level and down to a wholesale-aggregator level). Such functionality may also be provided at the transaction level, according to embodiments.

The system may be configured to allow de-duplication logic to be opted out per portal, in embodiments.

The system may be configured to provide reporting that details: total records that were received (per electronic medical record (EMR) vendor), how many records have had de-duplication logic applied to them (per EMR vendor), how many records have been removed from the response because of the de-duplication logic (per EMR vendor), and/or how many records were ultimately sent in the response back to the requesting EMR vendor, in embodiments.

Reporting details may include, without limitation, one or more of the following; Total count of all Medical History Requests received per EMR vendor—1000; Total count of Medical History Responses sent to EMR vendor—1000; Total count of Medical History Responses that had de-duplication logic applied to it per EMR vendor—800; Total count of medication history records sent in Response (at the Medication dispensed level) per EMR vendor—8000; Total count of Medical History records (at the medication dispensed level) that had de-duplication processing logic applied (where records were removed from the response)—5000 medications (fill records) per EMR vendor used for example only (5000/8000) or (800/1000); Total count of medication history records received from the PBM/Payer, (prior to any aggregation/de-duplication logic); The Medical History Records that were either Kept/Removed/Untouched Records per EMR vendor; Total count of records kept after de-duplication logic was applied per EMR vendor; Total count of records that were removed from the response per EMR vendor (this includes claim to claim and claim to fill duplicates that were removed); Total count of records that were not impacted by the de-duplication logic per EMR vendor (unmatched records); How many duplicate records were removed from all responses (sum of all electronic health records (EHRs)); Count of total number of Medical History records that had de-duplication process applied between two claims; Count of total number of Medical History records that had a de-duplication process applied between claim to fill; and/or How many times did the system remove something because it was a duplicate because it was Claim to claim versus how many times something was removed because it was Claim to Fill. Also add duplicate due to Claim to PDMP, Fill to PDMP, or any combination thereof.

This reporting data may be utilized via reportings, including visual representations of reports such as in Tableau, in embodiments.

The system may be configured to provide subscribers of Medication History service with access to all original Pharmacy prescription fill data, this will be a look at the data without any duplicate transactions being removed. The system may be configured to provide subscribers of Medication History reconciliation service with access to all original PBM Paid Claim data, this will be a look at the data without any duplicate transactions being removed. The system may be configured to provide subscribers of a Medication History reconciliation service with access to all original PDMP data; in embodiments, such data may be provided without duplicate transactions being removed. The system may be configured to provide the ability to track any Medication History record that is removed (from a medication history response message) because it has been classified as a duplicate Medication History record.

The system may be configured to provide an indication to the requesting provider vendor of any Medical History record that had de-duplication processing applied to it (the data that was maintained in the Medical History response level, when a duplicate of that record had been removed due to the de-duplication process). This indication does not have to be included in the response back to the EMR vendor, in embodiments, e.g., customers may be provide with indicia that something has been de-duplicated.

The system may be configured to require that a combination of some or all of the below data is present (or can be inferred from direct data sources, e.g., via augmentation, as described herein) and matches exactly for de-duplication criteria: Dispensing Pharmacy—NCPDP (National Council for Prescription Drug Programs) identifier (ID); Drug Identifier—NDC (national drug code), UPC (universal product code), or RxNorm from the Unified Medical Language System® (UMLS®); Prescriber Identifier—NPI (National Provider Identifier); and/or Date—Fill/Written/Dispensed.

The system may be configured to require that a combination of some or all of the below data is present (or can be inferred from direct data sources) and meets a reasonable confidence interval to be a match for de-duplication criteria: Dispensing Pharmacy—NCPDP ID, Pharmacy name and address; Drug Identifier—Drug Description, NDC/UPC/RxNorm; Prescriber Identifier—Name, Clinic, Address, NPI; and/or Date—Fill/Written/Dispensed. As one non-limiting example, if a fill date in one record is Jul. 1, 2018 but the fill date in another record is Jul. 2, 2018 due to a timing difference between when the medication is filled versus when the pharmacy submits the claim to insurance, a reasonable confidence interval is determined (e.g., within 1 day), in embodiments.

For Claim to Claim de-duplication, the system may be configured to also ensure the same PBM's data is being matched with the exact same PBM data.

The system may be configured to provide the ability to remove/drop duplicate Medication History transactions found in paid prescription claims, PDMP data, and Pharmacy prescription fill data, prior to sending a Medical History response back to the requesting EMR vendor. Determining duplicate Claim to Claim Medication History transactions may be based on the below example matching criteria and must be present in both claim columns (, e.g., in the below table) to indicate a match (duplicate medical record).

The Claim to Claim scenario can occur when a Medical History request has been sent for a time period where the number of medications in the request exceeds a given or set number of medications. The requestor may receive an indication, e.g., “AQ,” denoting there are more medications available for this patient. The requestor then needs to make a second request. For example, a response sends back the first 300 medications that are from a period of time Jun. 5, 2018-Mar. 15, 2018. The next response would need to start from Mar. 15, 2018-Jan. 1, 2018 to get remaining the medications. Since the end date of the first request and start date of the second overlap (Mar. 15, 2018) there will be a duplicate record(s) that should be de-duplicated. The duplicate record(s) would only be removed after the process has been completed.

The system may be configured to match the following fields in a claim: Patient ID, PBM ID, Pharmacy NCPDP ID, Prescriber NPI, Last Fill Date, and/or NDC. In embodiments, if those fields all match then the system will identify that specific medical record as a duplicate record. The host Medical History may keep the first record received from the PBM and add it to the med reconciliation response.

All data elements in the Medication Dispensed record must be an exact match, and if not, then the record cannot be considered a duplicate, according to embodiments. Table 1 and Table 2 are shown below for illustration

TABLE 1

| | Paid Prescription Claims matched against | |
|---|---|---|
| | other Paid Prescription Claims. (SAME PBM) | |
| | Claim data | Claim data |
| | found 1st | found 2nd |
| | request | request |
| Patient Demographics | ASSUMED | ASSUMED |
| (MPI does the matching) | | |
| Pharmacy → NCPDP ID | REQUIRED | REQUIRED |
| Prescriber → NPI | REQUIRED | REQUIRED |
| ONE OF THESE DATE, | | |
| DATA ELEMENTS | | |
| Last Fill Date | REQUIRED | REQUIRED |

If any of the below date elements are available and FILL DATE is not, then these may be used to match Medical History records.

TABLE 2

| | | |
|---|---|---|
| Written Date | X | X |
| Date Picked Up/Dispensed | X | X |
| Date | | |
| Date Sold | X | X |
| Quantity Dispensed | X | X |
| Medication → NDC | REQUIRED | REQUIRED |
| PBM - Must be the same | REQUIRED | REQUIRED |
| PBM | | |

In embodiments, additional rules for prescriber matching may be used. For instance, if no NPI was sent by the pharmacy, then a SPI (Surescripts Provider Identifier) may be used as a crosswalk in the Directory to find the NPI; if no SPI was sent by the pharmacy, then a DEA (Drug Enforcement Administration) number may be used as a crosswalk in the Directory to find the NPI; if no DEA number was sent by the pharmacy, then a Last Name may be used if all other data elements (Patient, Pharmacy, Medication) match; etc.

Determining duplicate Claim to Claim Medication History transactions will be based on the below matching criteria and must be present in both claim columns (in the below table) to indicate a match (duplicate medical record).

The Claim to Claim scenario can occur when a Medical History request has been sent for a time period where the number of medications in the request exceeds a number of medications. The requestor may receive an “AQ” denoting there are more medications available for this patient. The requestor then needs to make a second request, e.g., a response sends back the first 300 medications that are from a period of time Jun. 5, 2018-Mar. 15, 2018. The next response would need to start from Mar. 15, 2018-Jan. 1, 2018 to get remaining medications. Since the end date of the first request and start date of the second overlap (Mar. 15, 2018) there will be a duplicate record(s) that should be de-duplicated. The duplicate record(s) would only be removed after the process has been completed.

The system may be configured to be configured to match the following fields in a claim: Patient ID, PBM ID, Pharmacy NCPDP ID, Prescriber NPI, Last Fill Date, and/or UPC. In embodiments, IF those fields all match then the system will identify that specific medical record as a duplicate record. The host Medical History will keep the first record received from the PBM and add it to the med reconciliation response.

Table 3 and Table 4 are shown below for illustration.

TABLE 3

| | Paid Prescription Claims matched against | |
|---|---|---|
| | other Paid Prescription Claims. | |
| | Claim data | Claim data |
| | found 1st | found 2nd |
| | request | request |
| Patient Demographics (MPI | ASSUMED | ASSUMED |
| does the matching) | | |
| Pharmacy → NCPDP ID | REQUIRED | REQUIRED |
| Prescriber → NPI | REQUIRED | REQUIRED |
| ONE OF THESE DATE, | | |
| DATA ELEMENTS | | |
| Last Fill Date | REQUIRED | REQUIRED |

TABLE 4

| If any of the below date | Paid Prescription Claims matched against | |
|---|---|---|
| elements are available and | other Paid Prescription Claims. | |
| FILL DATE is not, then these | Claim data | Claim data |
| may be used to match Medical | found 1st | found 2nd |
| History records. | request | request |
| Date Picked Up | X | X |
| Written Date | X | X |
| Date Sold | X | X |
| Quantity Dispensed | X | X |
| Medication/Supply → UPC | REQUIRED | REQUIRED |
| PBM - Must be the same | ASSUMED | ASSUMED |
| PBM | | |

When the paid prescription claim data to paid prescription claim data de-duplication process determines a duplicate exists, the system may be configured to keep the first claim received and remove the second duplicate claim.

In embodiments for determining duplicate paid prescription claims against Pharmacy prescription dispensing data, determinations may be based on the below matching criteria and must be present in both the Pharmacy and Paid Prescription claim columns (in the below table) to indicate a match (duplicate medical record).

The system may be configured to match the following fields from Pharmacy Dispensing data and Paid prescription claims: Patient ID, Pharmacy NCPDP ID, Prescriber NPI, Last Fill Date, and/or NDC (11). IF those fields all match then the system will identify that specific medical record as a duplicate record. The host Medical History may keep the first record received from the PBM and add it to the med reconciliation response.

A pharmacy fill record or a PDMP data record can possibly match to two claim records and the system may be configured to drop the claim records and maintain the pharmacy fill record or the PDMP data record.

Additional “fuzzy logic” may be used to determine likely matches—i.e. if last fill date is off by +/−24 hours, this would be considered a match.

TABLE 5

| | | |
|---|---|---|
| Patient Demographics (MPI | ASSUMED | ASSUMED |
| does the matching) | | |
| Pharmacy → NCPDP ID | REQUIRED | REQUIRED |
| Prescriber → NPI | REQUIRED | REQUIRED |
| Last Fill Date (required to be | REQUIRED | REQUIRED |
| sent in at least one loop | | |
| from the PBM) | | |

IF any of the below date elements are available, then also use them to match Medical History records

TABLE 6

| | | |
|---|---|---|
| Date Picked Up | N/A | N/A |
| Written Date | N/A | N/A |
| Date Sold | N/A | N/A |
| Quantity Dispensed | N/A | N/A |
| Medication/Supply → NDC | REQUIRED | REQUIRED |
| PBM | N/A | N/A |

TABLE 7

| | Pharmacy Prescription Dispensing Data | |
|---|---|---|
| | matched against Paid Prescription Claims/PDMP | |
| | Pharmacy | Paid |
| | Prescription Fill | Prescription |
| | Data | Claim Data |
| Patient | ASSUMED | ASSUMED |
| Demographics (MPI | | |
| does the matching) | | |
| Pharmacy → NCPDP | REQUIRED | REQUIRED |
| ID | | |
| Prescriber → NPI | REQUIRED | REQUIRED |
| Last Fill Date | REQUIRED | REQUIRED |
| (required to be sent | | |
| in at least one loop | | |
| from the PBM) | | |

IF any of the below date elements are available, then also use them to match Medical History records

TABLE 8

| | Pharmacy Prescription Dispensing Data | |
|---|---|---|
| | matched against Paid Prescription Claims | |
| | Pharmacy | Paid |
| | Prescription Fill | Prescription |
| | Data | Claim Data |
| Date Picked Up | N/A | N/A |
| Written Date | N/A | N/A |
| Date Sold | N/A | N/A |
| Quantity Dispensed | N/A | N/A |
| Medication/Supply | REQUIRED | REQUIRED |
| → UPC (this assumes | | |
| that both | | |
| pharmacies and | | |
| PBM's use UPC for a | | |
| supply) | | |
| PBM | N/A | N/A |

TABLE 9

| | | |
|---|---|---|
| Patient Demographics (MPI | ASSUMED | ASSUMED |
| does the matching) | | |
| Pharmacy → NCPDP ID | REQUIRED | REQUIRED |
| Prescriber → NPI | REQUIRED | REQUIRED |
| Last Fill Date (required to | REQUIRED | REQUIRED |
| be sent in at least one loop | | |
| from the PBM) WHERE | | |
| there is a difference of +/−1 | | |
| day between Fill & Claim | | |
| dates | | |

IF any of the below date elements are available, then also use them to match Medical History records.

TABLE 10

| | | |
|---|---|---|
| Date Picked Up | N/A | N/A |
| Written Date (Exact date | REQUIRED | REQUIRED |
| match) | | |
| Date Sold | N/A | N/A |
| Quantity Dispensed | | |
| Medication/Supply → NDC | REQUIRED | REQUIRED |
| PBM | N/A | N/A |

TABLE 11

| | | |
|---|---|---|
| Patient Demographics (MPI | ASSUMED | ASSUMED |
| does the matching) | | |
| Pharmacy → NCPDP ID | REQUIRED | REQUIRED |
| Prescriber → NPI | REQUIRED | REQUIRED |
| Last Fill Date (required to be | REQUIRED | REQUIRED |
| sent in at least one loop | | |
| from the PBM) WHERE there | | |
| is a difference of +/−1 day | | |
| between Fill & Claim dates | | |

IF any of the below date elements are available, then also use them to match Medical History records.

TABLE 12

| | | |
|---|---|---|
| Date Picked Up | N/A | N/A |
| Written Date (Exact match) | REQUIRED | REQUIRED |
| Date Sold | N/A | N/A |
| Quantity Dispensed | N/A | N/A |
| Medication/Supply → UPC | REQUIRED | REQUIRED |
| PBM | N/A | N/A |

TABLE 13

| | | |
|---|---|---|
| Patient Demographics (MPI | ASSUMED | ASSUMED |
| does the matching) | | |
| Pharmacy → NCPDP ID | REQUIRED | N/A |
| Prescriber → NPI | REQUIRED | REQUIRED |
| Last Fill Date (required to | IS | N/A |
| be sent in at least one loop | PRESENT | |
| from the PBM) | | |

IF any of the below date elements are available, then also use them to match Medical History records.

TABLE 14

| | | |
|---|---|---|
| Date Picked Up | | |
| Written Date | N/A | IS |
| | | PRESENT |
| Date Sold | | |
| Quantity Dispensed | REQUIRED | REQUIRED |
| Medication/Supply | REQUIRED | REQUIRED |
| → UPC (this assumes that | | |
| both pharmacies and PBM's | | |
| use UPC for a supply) | | |
| PBM | ASSUMED | ASSUMED |

IF any of the below date elements are available, then also use them to match Medical History records.

TABLE 15

| | | |
|---|---|---|
| Patient Demographics (MPI | ASSUMED | ASSUMED |
| does the matching) | | |
| Fill Date (Exact date match) | REQUIRED | REQUIRED |
| Pharmacy → NCPDP ID | REQUIRED | REQUIRED |
| Medication/Supply → Drug | REQUIRED | REQUIRED |
| Description | | |
| Medication/Supply → | REQUIRED | N/A |
| Compendia Med ID | | |
| PBM | N/A | N/A |

IF any of the below date elements are available, then also use them to match Medical History records.

TABLE 16

| | | |
|---|---|---|
| Patient Demographics (MPI | ASSUMED | ASSUMED |
| does the matching) | | |
| Fill Date (Exact date match) | REQUIRED | REQUIRED |
| Pharmacy → NCPDP ID | REQUIRED | REQUIRED |
| Medication/Supply → NDC | N/A | N/A |
| Prescription Number | REQUIRED | REQUIRED |
| PBM | N/A | N/A |

TABLE 17

| | | |
|---|---|---|
| Patient Demographics (MPI | ASSUMED | ASSUMED |
| does the matching) | | |
| Pharmacy → Address | REQUIRED | N/A |
| Information | | |
| Prescriber → NPI | REQUIRED | REQUIRED |
| Last Fill Date (required to be | IS | N/A |
| sent in at least one loop | PRESENT | |
| from the PBM) | | |

### B. Example Data Augmentation Embodiments

In embodiments, data augmentation is performed. Improving medication history data through augmenting any unpopulated data fields with data from other approved sources is contemplated according to such embodiments. In some embodiments, data augmentation may be performed subsequent to the performance of data de-deduplication, as described herein. Embodiments also provide for generating a correct and complete label/description (i.e., a “sig” or “SIG”) for a prescription data field based on natural language text provided therefor, as well as calculating and populating dosage data fields, e.g., for equivalent or substitute drugs. Augmentation may be performed for Medication History for Reconciliation (MHR), Medication History for Ambulatory (MHA), Medication History for Long Term Post Acute Care (MH-LTPAC) transactions, and/or the like.

In an example, a method for data augmentation may be performed by a system or device configured to perform one or more operations thereof. For instance, a provider vendor, e.g., a doctor or doctor office staff member, sends a request for a prescription history (“RxHistoryRequest”) for an eligible patient. The host system validates the message, passes it to the appropriate PBM/payer and looks for pharmacy fill data in the pharmacy database. The host then validates the level of consent as Yes or No. The host may then check for the date range of the history request. If populated with a Start and End date, a number of medications may be returned to the requestor, e.g., starting with the End date, in embodiments. A PBM/payer may the process the RxHistoryRequest and check for the date range of the history request. The PBM/payer may validate the level of consent as Yes, and if populated with a Start and End date, a number of medications may be returned to the host, e.g., starting with the end date. The PBM/payer may create a response for the prescription history request (“RxHistoryResponse”) and submit it back to the host where it is validated. Similarly, a PDMP may also receive and process the RxHistoryRequest and provide an appropriate RxHistoryResponse. The host system may then aggregate Pharmacy Fill data, PDMP data, and/or PBM paid claim data, and generate and send the RxHistoryResponse to the originating requestor. The host may then aggregate Pharmacy Fill data, PDMP data, and PBM paid claim data, and then generate and send the Medication History response to the originating requestor.

The aggregating step above may include augmentation operations, in embodiments. For example, the host system may be configured to identify data elements that are eligible for augmentation processing and/or that have not already been populated by the data suppliers (e.g., pharmacy, PDMP, and/or PBM) and the EHR provider has “Opted In” to receive augmented data. The host system may be configured to then use the appropriate identified data source to augment the data elements found in each unique medication dispensed occurrence (host directory services, and/or host-downloaded versions of commercial and/or government drug compendia). The host system may be configured to track each medication dispensed occurrence that has been augmented (so that the provider vendor can identify what has been augmented either at the RxHistoryResponse level, at the individual medication dispensed level, and/or at the data element level).

In data augmentation embodiments, the system may be configured to provide the ability to augment existing Medication History Response messages with data from other data sources. For example, the system may be configured to use key data to match key data within other data sources, to ensure that the correct values are being augmented. Key data may be, without limitation, one or more of the following:

Drug identifier such as an NDC (11 digit value);

Pharmacy identifier such as an Pharmacy NCPDP (or NPI);

Prescriber identifier such as an Prescriber NPI (e.g., an SPI (Surescripts Provider Identifier));

Drug information (drug description, strength, form, class, etc.);

Codified patient directions;

Codified notes made by a prescriber or pharmacy; and/or

Diagnosis codes entered in patient directions or other free text fields.

The system may, as noted above, be configured to provide augmented data to medication history entities, in embodiments. The system may be configured to provide augmented data only when the data for a specific data element is not supplied by the pharmacy, PDMP, or PBM data suppliers, in embodiments. Similarly, augmentation may take place at the individual data element level, i.e., prescriber First Name, or Prescriber Last Name or Pharmacy street address, and/or the like, in embodiments.

The system may be configured to provide augmented data in the Medical History response to the originating requestor and may not store the augmented data in a local database. The system may also be configured to provide the ability to only allow approved/subscribing customer access to the augmented Medical History data.

The system may be configured to provide the ability to “Opt In” and/or “Opt Out” to this augmented Medical History data. This “Opt In”/“Opt Out” functionality may be provided at a sub account (portal) level and at a Health care provider system level (e.g., down to health system level and down to a wholesale-aggregator level). In embodiments, the “Opt In”/“Opt Out” functionality may be provided/configured at the transaction level. All Health care systems may have to decide to “Opt In” upon initial setup in order to receive augmented data, in embodiments.

The system may be configured to provide internal reporting that provides details about the following: data element name, source of the augmented data (a directory, drug compendia, etc.), when augmenting data. When unable to augment the data for fields that have met the criteria for augmentation, the system may be configured to provide the following: data element name, expected source for augmenting, associated with the data element, and a reason for not populating, e.g., data element not populated at the source (a directory, drug compendia, etc.), system error (e.g., service is down), data provided by the source is invalid (e.g., does not pass validity checks or key data given was bogus and not able to be found), etc.

The system may be configured to track augmented data in embodiments by specific Pharmacy organization, PDMP entity, or PBM/Payer organization to include the following, without limitation: Count and store the number of data elements that could be augmented by data supplier, Number of elements augmented, percentage of augmented data.

The system may be configured to track augmented data in embodiments by total Pharmacy, Total PBM/Payer or state PDMP to include the following, without limitation: Count and store the number of data elements that could be augmented by data supplier, number of elements augmented, percentage of augmented data, and/or count of transactions augmented.

The system may be configured to may track augmented data in embodiments by total EMR vendors to include the following, without limitation: Count and store the number of data elements that could be augmented by data supplier, number of elements augmented, percentage of augmented data, and/or count of transaction augmented.

The system may be configured to provide the ability for a requestor to view the original Medical History fill file data, prior to any data being augmented. The system may be configured to track data that has been augmented at the individual data element level or at the message level, so that EMR vendors/entities involved in the data transactions are enabled to understand the data was augmented. Tracking for augmented data may be provided in the actual response message or as an indicator that data was augmented somewhere in the message and coupled with provided details in another format, in embodiments. For example, text details of what data has been augmented may be provided at the Dispensed Medication level in the “Note” field. In embodiments, an indication of the augmentation process may be identified at the Medical History Response Level or at the Medication Dispensed Level.

Embodiments herein also contemplate augmenting via calculated fields, for instance, using fields sent in the medication information along with additional data sources, as reference data, to calculate dosage information, opioid risk scores, ascertain diagnosis codes, highlight data discrepancies, calculate a number of refills remaining, calculate cancel dates, flag records which may need advanced review, etc. For instance, a morphine equivalent drug may have been prescribed to a patient where the dosage units were not included in the patient's record or prescription history information. Embodiments herein provide for identifying the data field for the dosage units as being eligible for augmentation, e.g., by identifying the field as blank or unpopulated, determining the equivalent drug identifier from an associated data field, retrieving information about the equivalent drug and/or information about morphine dosage units, and calculating dosage units for the equivalent drug as the augmenting data.

Embodiments herein also contemplate augmenting existing fields that are populated with data, in addition to embodiments noted above that refer to adding data to fields where they are blank when received from the provider. In embodiments, augmenting existing fields includes changing or improving the data in an existing field. In embodiments, augmenting existing fields may be performed responsive to a mismatch in data elements, or to poor quality or missing data within the field that can be added using a drug compendia source. A host system is configured to replace and/or modify the data field in question to correct or supplement the field data to comply with standards, be technically accurate, improve quality of data/description, etc. As anon-limiting example, a drug description field may be received that includes “<DrugName>10,” and the host system is configured to augment the drug description field to “<DrugName>10 mg Tablet.” Additional examples include, without limitation, augmenting existing fields for standardizing drug descriptions, adding leading zeros to medication strengths, removing trailing zeros from medication strength, removing reference drugs from drug description, adding strength units when missing, removing or replacing abbreviations, etc. Augmenting existing fields may be performed for multiple fields including, but not limited to, drug descriptions, free text SIGs, and/or provider notes. Sources for augmenting existing fields with data may include compendia, as described herein.

Embodiments herein also contemplate aspects for utilizing Structured SIG. For example, systems herein may be configured to interpret free text or natural language text in patient directions (SIG) fields to generate codified elements (e.g., per the NCPDP Standard) from the free text or natural language text, e.g., as codes for dosage, form, etc. Such embodiments are configured to use referential matching of a list of example free text or natural language, along with associated codes to train a corresponding neural network model, described in further detail below, to approximate the relevant structured code with a desired degree of certainty. Such models are configured to generalize the text string even if there are inconsistencies, and to create additional sample text strings along with codes for human validation to continue to train the algorithm over time with additional validated human confirmed strings. Validated strings may come from a proprietary source (i.e., validated by clinicians associated with the host system) or may come from a third party source(s) (e.g., compendia, standards organizations, etc.). Classification of each data element may be done using a BERT (Bidirectional Encoder Representations from Transformers) model, or the like, modified for classification tasks for each code type classified by the system. In embodiments, the BERT model is utilized to classify free text into separate codes by adding an output layer(s) specific to the number of possible types or classes (e.g., route of administration codes, etc.) that are desired to identify in the output. A confidence threshold may be set for each element to quantify certainty for the elements, and in embodiments, the threshold is configurable or dynamically configurable based on the code and/or the code type. For instance, in one example scenario, a code for “Oral Route” is provided back if the machine learning model is >98%, for example, certain that is the correct code.

In embodiments, models are re-trained and/or updated offline based on accumulated training data, and are not dynamically updated when new data is received by the system. That is, models may be persisted in deployment for extended periods of time between trainings and re-deployments.

Table 18 below shows medication data augmentation examples, including message field names, augmentation sources, and connecting data points.

TABLE 18
Example Medication Data Augmentation

| | | | | |
|---|---|---|---|---|
| PrescriberIDDEA | Directory | DEANumber | NPI (SPI is | IF the Doctor |
| | | | preferred, will | has a controlled |
| | | | identify the | substance |
| | | | Doctor, BUT | service level, |
| | | | NOT the | THEN this |
| | | | location | element is |
| | | | where the | required. i.e, |
| | | | doctor is | 100% of the |
| | | | prescribing | prescribers on |
| | | | for a specific | the SS network, |
| | | | patient | who prescribe |
| | | | interaction) | controlled |
| | | | | substances, have |
| | | | | a DEA number. |
| PrescriberLastName | Directory | Last Name | NPI | |
| PrescriberFirstName | Directory | First Name | NPI | |
| PrescriberPhoneNumber | Directory | Telephone | NPI | This is |
| | | (primary) | | considered an |
| | | | | instance of |
| | | | | Primary Phone |
| | | | | Number at a |
| | | | | specific address. |
| | | | | However, a |
| | | | | prescriber can |
| | | | | have multiple |
| | | | | address per NPI. |
| PrescriberFaxNumber | Directory | Fax | NPI | This is |
| | | | | considered an |
| | | | | instance of |
| | | | | Primary Phone |
| | | | | Number at a |
| | | | | specific address. |
| | | | | However, a |
| | | | | prescriber can |
| | | | | have multiple |
| | | | | address per NPI. |
| PharmacyDEA | Directory | DEA | NCPDP ID | |
| | | Number | | |
| PharmacyNPI | Directory | NPI | NCPDP ID | |
| PharmacySpecialty | Directory | Specialty | NCPDP ID | This will |
| | | | | indicate if a |
| | | | | pharmacy is a |
| | | | | Specialty, Retail |
| | | | | or Mail Order |
| PharmacyName | Directory | Organization | NCPDP ID | |
| | | Name | | |
| PharmacyAddress1 | Directory | Address Line | NCPDP ID | |
| | | 1 | | |
| PharmacyCity | Directory | City | NCPDP ID | |
| PharmacyState | Directory | State | NCPDP ID | |
| PharmacyPostalCode | Directory | Zip | NCPDP ID | |
| PharmacyPrimaryPhoneNumber | Directory | Telephone | NCPDP ID | |
| | | (primary) | | |
| PharmacyFaxNumber | Directory | Fax | NCPDP ID | |
| DoseDeliveryMethod | Calculation | Dose | SigText | |
| | | Delivery | | |
| | | Method | | |
| DoseQuantity | Calculation | Quantity | SigText | |
| DoseUnitOfMeasure | Calculation | Unit of | SigText | |
| | | Measure | | |
| RouteOfAdministration | Calculation | Route | SigText | |
| Frequency | Calculation | Frequency | SigText | |
| FrequencyUnits | Calculation | Frequency | SigText | |
| | | Units | | |
| AdministrationTiming | Calculation | Timing | SigText | |
| Instructionindicator | Calculation | Instructions | SigText | |
| IndicationPrecursor | Calculation | Indication | SigText | |
| IndicationValue | Calculation | Indication | SigText | |
| DurationNumericValue | Calculation | Duration | SigText | |
| | | Amount | | |
| DurationText | Calculation | Duration | SigText | |
| | | Unit | | |
| Medication Name | Drug | Name of | NDC or drug | a standardized |
| | Compendia | medication | description | 30-character |
| | | dispensed | | alphanumeric |
| | | | | column that |
| | | | | contains a |
| | | | | combination of |
| | | | | the drug name |
| | | | | appearing on |
| | | | | the package |
| | | | | label, the |
| | | | | strength |
| | | | | description, and |
| | | | | the dosage form |
| | | | | description for a |
| | | | | specified |
| | | | | product. |
| StrengthValue | Drug | Numerical | NDC or drug | Ex: 10 for 10 mg |
| | Compendia | value of | description | |
| | | strength of | | |
| | | medication | | |
| StrengthFormCode | Drug | Alphanumeric | NDC or drug | EXAMPLES: |
| | Compendia | code | description | C42966 - |
| | | representing | | Ointment |
| | | the form of | | C25158 - |
| | | the | | Capsule |
| | | medication | | C42994 - |
| | | | | Suspension |
| | | | | C42998 - Tablet |
| StrengthUnitOfMeasureCode | Drug | Alpha value | NDC or drug | i.e. C48155 = |
| | Compendia | of the | description | Gram |
| | | strength | | C28254 = |
| | | units of the | | Mililiter |
| | | medication | | |
| QuantityUnitOfMeasure | Drug | Alphanumeric | NDC or drug | i.e. Gram, |
| | Compendia | code | description | Mililiter |
| | | representing | | |
| | | the | | |
| | | description | | |
| | | of strength | | |
| | | units of the | | |
| | | medication | | |
| Drug Enforcement | Drug | a one- | NDC or drug | Ex: C |
| Administration Code | Compendia | character | description | |
| | | alphanumeric | | |
| | | value that | | |
| | | identifies a | | |
| | | drug's | | |
| | | federal | | |
| | | controlled | | |
| | | substance | | |
| | | schedule and | | |
| | | potential for | | |
| | | abuse. This | | |
| | | code is | | |
| | | subject to | | |
| | | change by | | |
| | | federal | | |
| | | regulation- | | |
| Labeler | Drug | a six- | NDC or drug | Ex: A00002 = ELI |
| | Compendia | character | description | LILLY & CO |
| | | alphanumeric | | |
| | | column | | |
| | | that contains | | |
| | | a code | | |
| | | assigned by | | |
| | | drug | | |
| | | compendia | | |
| | | to uniquely | | |
| | | identify the | | |
| | | product | | |
| | | labeler (a | | |
| | | manufacturer, | | |
| | | distributor, | | |
| | | or | | |
| | | repackager). | | |

### C. Example System and Operational Embodiments

As described herein with respect to embodiments, clinical information, medication information, etc., may be exchanged between computer systems as part of transactions and/or requests for information in which data may be de-duplicated and/or augmented. For instance, in FIG. 1, a block diagram of a network of computer systems 100 that includes a de-duplicator 104 and an augmenter 106 is shown, according to an embodiment. Network of computer systems 100 includes a host server 102 that may include one or more processing devices such as, but not limited to, servers, and a remote computer system(s) 110 that may also include one or more processing devices such as, but not limited to, servers and client devices such as laptop/desktop computers and computer terminals, personal handheld devices, etc. Host server 102 may be communicatively coupled or linked to requestor system 106 via a communication link 108.

Host server 102 may comprise one or more computers/servers of a host entity facilitating access to de-duplicator 104 and/or augmenter 106 by remote computer system(s) 106, according to embodiments. Host server 102 may include geographically distributed computers/servers, a rack server system(s), a stand-alone server(s), etc.

Remote computer system(s) 106 may comprise one or more computers/servers of an entity, such as a trading partner(s), a vendor service(s), a doctor or doctor's office (including nurses and/or other staff), a pharmacy, a PBM, an MHR, and/or the like as noted herein, that desires to request medication information for patients from host server 102 over communication link 108.

Communication link 108 may comprise at least one network or direct communication connection, or any combination thereof, between host server 102 and remote computer system(s) 106 that enables communication messages such as requests/responses for medication information of patients, as described herein, along with any associated messages required for data de-duplication and/or augmentation. As used herein, the term “messages,” “communication messages,” etc., includes without limitation resources such as clinical resources, data, information, packets, and/or the like, related to messaging such as clinical messaging, transmitted and/or received according to any communication standard or protocol, or according to ad hoc communications. In embodiments, communication link 108 may comprise wired and/or wireless portions of one or more networks such as networks of the host entity and requestors, including enterprise networks, the Internet, etc.

De-duplicator 104 and/or augmenter 106 may comprise hardware and/or software components configured to perform operations for de-duplication and/or augmentation, respectively, as described herein.

It is contemplated herein, according to embodiments, that host server 102 may comprise one or more servers that perform the described functionality of de-duplicator 104 and/or augmenter 106, as well as other functionality for a host entity, or may be a single server performing either or both of these types of functionality. Other relational configurations of host server 102 are also contemplated herein, as would be understood by a person of skill in the relevant art(s) having the benefit of this disclosure.

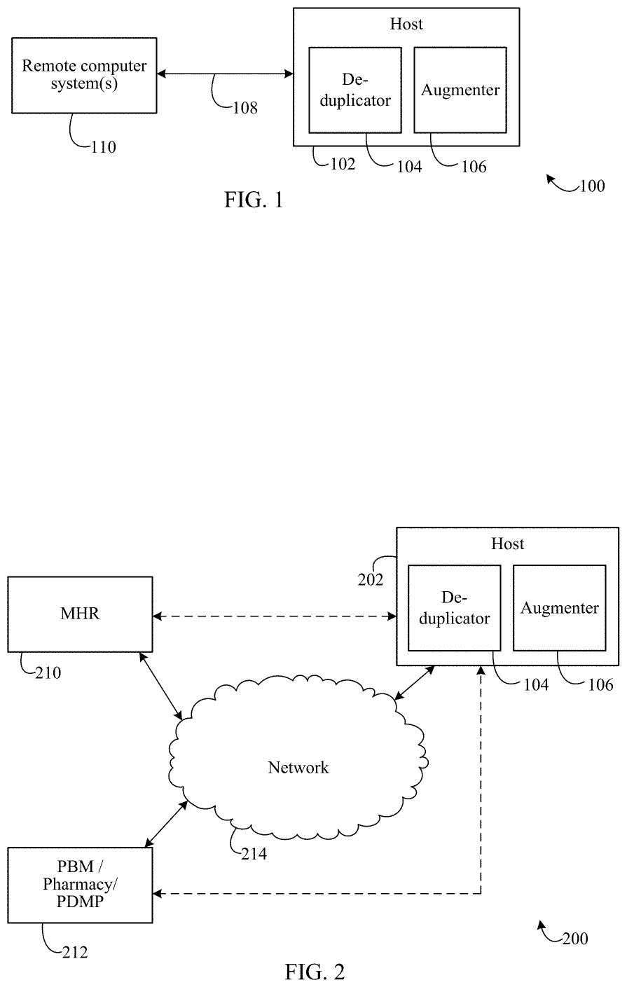

Turning now to FIG. 2, a block diagram of a network of computer systems 200 that includes de-duplicator 104 and augmenter 106 is shown, according to an embodiment. Network of computer systems 200 may be a further embodiment of network of computer systems 100 of FIG. 1. Network of computer systems 200 includes a host server 202, an MHR system 210, and a PBM system 212. Host provider 202, MHR system 210, and PBM system 212 may be communicatively coupled or linked each other via a network 214. MHR system 212 represents one or more systems in embodiments, including but not limited to, MHR systems, MHA systems, MH-LTPAC systems, and/or the like. PBM system 212 represents one or more systems in embodiments, including but not limited to, PBM systems, pharmacy systems, PDMP systems, and/or the like.

Host server 202 may be a further embodiment of host server 102 of FIG. 1, and, for the purposes of illustration for FIG. 2, is configured the same, or substantially the same, as host server 102 above. Network 214 may be a further embodiment of communication link 108 of FIG. 1. Network 214 may comprise at least one network and/or direct connection (i.e., a communication link), or any combination thereof. That is, network 214 may be any combination of the Internet, the “cloud,” direct communication links, business and/or enterprise networks, and/or the like.

As noted, network 214 is configured to communicatively couple host server 202, MHR system 210, and PBM/Pharmacy/PDMP system 212 to each other. Accordingly, network of computer systems 200 is configured as a further embodiment of network of computer systems 100 in that computer systems 200 is configured to perform data de-duplication and/or data augmentation for medication information requests as described herein.

With respect to FIG. 2, while shown for illustrative simplicity and brevity as including a single host (e.g., host server 202) and two remote computer systems (MHR 210 and PBM 212), it is contemplated herein that network of computer systems 200 may include more or fewer of any of these components, as well as different types of remote systems described herein, in embodiments. In embodiments, MHR 210 and/or PBM 212 may include a database of associated information, e.g., prescription fill data, PDMP data, and/or the like, as described herein.

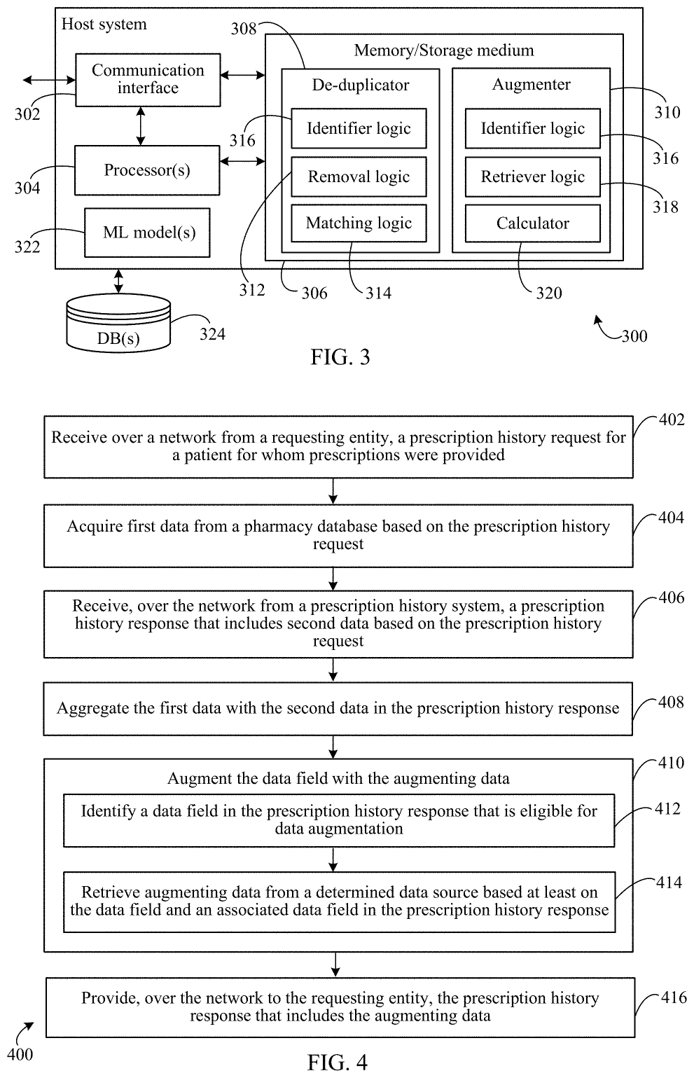

Turning now to FIG. 3, a block diagram of a host system 300 configured to perform operations for data de-duplication and medication data augmentation is shown, according to an embodiment. Host system 300 may be a further embodiment of host server 102 of FIG. 1 and/or host server 202 of FIG. 2. That is, host system 300 may be included or implemented in a host server, e.g., host server 102 of FIG. 1 and/or host server 202 of FIG. 2, that is communicatively coupled to one or more remote computer systems over a communication link or network, as described herein.

Host system 300 includes a communication interface 302, one or more processors (“processor”) 304, and a memory/storage medium (“memory”) 306. Processor 304 is communicatively coupled to communication interface 302 and to memory 306. Communication interface 302 is configured to be communicatively coupled to a communication link and/or to a network, such as communication link 108 of FIG. 1 and/or network 210 of FIG. 2 for communication with one or more remote devices such as requestors as described herein, by which host system 300 can receive, acquire, and/or retrieve information from other systems described herein, including records and other database information. Communication interface 302 may be one or more interfaces, such as hardware network interfaces, that are configured to transmit and/or receive communications and messages over a network or communication link as described herein.

Processor 304 may be one or more computer processors or processing devices as known to one of skill in the relevant art(s) having the benefit of this disclosure, such as those configured to operate in a computer, a server, a computing systems, and/or the like, as described herein. Processor 304 is configured to execute computer program instructions to perform the described data de-duplication and/or data augmentation functions and methods.

Memory 306 is a hardware device(s) of, or associated with, a computer, a server, a computing system, and/or the like, as described herein, that is configured to store data/information and/or computer program instructions that may be executed by processor 304. For example, as shown in FIG. 3, memory 306 is configured to store de-duplicator logic 308 and augmenter logic 310. Memory 306 is also configured to store machine learning (ML) model(s) 320, in embodiments, that are utilized as described herein. Memory 306 is also configured to store data and/or information received over a network from remote systems, including but not limited to, prescription history requests, prescription history responses, augmenting data, reference data, a host directory, drug compendia, and/or the like, as described herein.

Host system 300 may also include one or more databases (DBs) 324, in embodiments. DB(s) 324 may include host directories, local-host versions of compendia, and/or any other database/records information described herein. In embodiments, DB(s) 324 may be internal or external to host system 300, and in some embodiments, may comprise a portion of memory 306.

De-duplicator logic 308 is illustrated as including removal logic 312 and matching logic 314. Additional components for performing aspects of data de-duplication are also contemplated as being included in de-duplicator logic 308, e.g., aggregator logic configured to aggregate data from different sources, in embodiments, but are not shown for illustrative clarity and brevity. Functions and/or operations of such components are contemplated as being performed by de-duplicator logic 308 when not explicitly described. Removal logic 312 may be configured to remove duplicate data and/or records herein, such as records included in RxHistoryResponse messages.

Matching logic 314 may be configured to determine matching, or near matching, between data and/or records, including for dates, date ranges, whole records or partial portions thereof, and/or the like, as described herein for embodiments. De-duplicator logic 308 may also include identifier logic 316 that is configured to identify data and/or records that are eligible for data de-duplication operations, as described herein.

Augmenter logic 310 is illustrated as including retriever logic 318 and calculator logic 320. Additional components for performing aspects of data augmentation are also contemplated as being included in augmenter logic 310, e.g., aggregator logic configured to aggregate data from different sources, in embodiments, but are not shown for illustrative clarity and brevity. Functions and/or operations of such components are contemplated as being performed by aggregator logic 310 when not explicitly described.

Retriever logic 318 is configured to retrieve data, e.g., from directories and/or compendia in DB(s) 324, as augmenting data, and calculator logic 320 is configured to generate numerical values based on associated data, e.g., dosage data and/or units, as described herein. Augmenter logic 310 may also include identifier logic 316 that is configured to identify data and/or records that are eligible for data augmentation operations, as described herein.

It should be noted that as described herein, embodiments are applicable to any type of system that communicates with other systems and/or devices over a network. One example is where a host system in a “cloud” network architecture/platform. A cloud platform includes a networked set of computing resources, including servers, routers, etc., that are configurable, shareable, provide data security, and are accessible over a network such as the Internet. Cloud applications run on the resources, often atop operating systems that run on the resources, for entities that access the applications over the network. A cloud platform may support multi-tenancy, where cloud platform-based software services multiple tenants, with each tenant including one or more users who share common access to software services of the cloud platform. Furthermore, a cloud platform may support hypervisors implemented as hardware, software, and/or firmware that run virtual machines (emulated computer systems, including operating systems) for tenants. A hypervisor presents a virtual operating platform for tenants.

FIG. 4 shows a flowchart 400 for data augmentation, according to example embodiments. The systems in FIG. 1, systems in FIG. 2, and/or host system 300 in FIG. 3 operate according to flowchart 400, in embodiments. Further structural and operational examples will be apparent to persons skilled in the relevant art(s) based on the following descriptions. Flowchart 400 is described below with respect to the systems in FIG. 1, systems in FIG. 2, and/or host system 300 in FIG. 3.

Flowchart 400 begins at step 402. In step 402, a prescription history request for a patient for whom prescriptions were provided is received over a network from a requesting entity. For instance, host system 300 is configured to receive a prescription history request (RxHistoryRequest) via communication interface 302 from a requestor, such as a health care provider, as described herein. In embodiments, host system 300 may be configured to validate the request, e.g., via de-duplication logic 308 and/or augmenter logic 310, and to forward or transmit the request, e.g., to a PBM such as PBM 212 in FIG. 2, for generation of a response. In embodiments, the request may include a date range in which records are sought.

In step 404, first data from a pharmacy database is acquired based on the prescription history request. For example, host system 300 is configured to acquire, by request and/or retrieval via retriever logic 318, data such as records from a pharmacy database related to filled prescriptions in the requested date range, as described herein.

In step 406, a prescription history response that includes second data based on the prescription history request is received, over the network, from a prescription history system. For instance, a RxHistoryResponse may be received by augmenter logic 310, via communication interface 302, from a PBM to which the request in step 402 was forwarded or transmitted. The response may include one or more records for transactions/filled prescriptions for the patient. In embodiments, a PDMP may be the prescription history system, and in some embodiments a prescription history system may comprise both a PBM and a PDMP where the second data may include data from each of the PBM and the PDMP.

In step 408, the first data is aggregated with the second data in the prescription history response. For example, augmenter 310 of host system 300 may be configured to aggregate data received from the PBM and the pharmacy database. In embodiments, this aggregation may be performed in the context of the response (RxHistoryResponse) in order to provide a complete response to the request.

In step 410, the data field is augmented with the augmenting data. Augmenting data fields as described herein may be performed by data augmenter logic 310 of host system 300. In embodiments, one or more fields in a record may be augmented, and multiple records in a response may be augmented. Step 410 may comprise one or more sub-steps in embodiments, e.g., step 412 and/or step 414, described below.

In step 412, a data field in the prescription history response that is eligible for data augmentation is identified. For instance, identifier logic 316 of augmenter logic 310 may be configured to identify data fields in the record(s) of the response that are eligible candidates for data augmentation. Candidate data fields may include free text sig fields, dosage fields, drug name or drug identifier fields, patient data fields, and/or the like, as noted in this description.

In step 414, augmenting data is retrieved from a determined data source based at least on the data field and an associated data field in the prescription history response. For example, retriever logic 318 may be configured to retrieve augmenting data to be used for augmentation, as described herein, from a local or remote data source. In embodiments, data in DB(s) 324 of FIG. 3 may comprise one or more data sources. Data sources may be determined by identifier logic 318 based on the data field, on associated data fields, etc., in the prescription history response.

In embodiments, calculator 320 of augmenter logic 310 is configured to calculate numerical values as augmenting data, as described above, where calculator 320 comprises a determined data source and the augmenting data is retrieved, including received, therefrom.

In some embodiments, augmenting data may not exist or may not be available. In such scenarios, in place of augmenting data, retriever logic 318 may be configured to provide one or more the following information instead: data element name, expected source for augmenting, associated with the data element, and a reason for not populating, e.g., data element not populated at the source (a directory, drug compendia, etc.), system error (e.g., service is down), data provided by the source is invalid (e.g., does not pass validity checks or key data given was bogus and not able to be found), etc.

In step 416, the prescription history response that includes the augmenting data is provided over the network to the requesting entity. For instance, augmenting data generated and/or obtained by augmenter logic 310 and its components may be provided with the RxHistoryResponse received in step 406 above back to the requesting entity via communication interface 302.

In embodiments, tracking data such as metadata, may also be provided with the prescription history response with the augmenting data in order to identify and track augmented data.

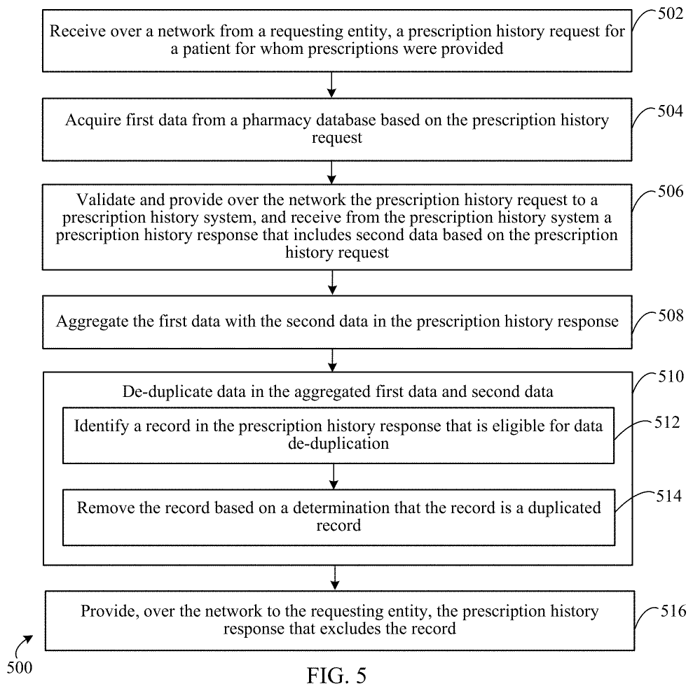

FIG. 5 shows a flowchart 500 for data de-duplication, according to example embodiments. The systems in FIG. 1, systems in FIG. 2, and/or host system 300 in FIG. 3 operate according to flowchart 500, in embodiments. Further structural and operational examples will be apparent to persons skilled in the relevant art(s) based on the following descriptions. Flowchart 500 is described below with respect to the systems in FIG. 1, systems in FIG. 2, and/or host system 300 in FIG. 3.

Flowchart 500 begins at step 502. In step 502, a prescription history request for a patient for whom prescriptions were provided is received over a network from a requesting entity. For instance, host system 300 is configured to receive a prescription history request (RxHistoryRequest) via communication interface 302 from a requestor, such as a health care provider, as described herein. In embodiments, host system 300 may be configured to validate the request, e.g., via de-duplication logic 308 and/or augmenter logic 310, and to forward or transmit the request, e.g., to a PBM such as PBM 212 in FIG. 2, for generation of a response. The request may include a date range in which records are sought.

In step 504, first data from a pharmacy database is acquired based on the prescription history request. For example, host system 300 is configured to acquire, by request and/or retrieval via retriever logic 318, data such as records from a pharmacy database related to filled prescriptions in the requested date range, as described herein.

In step 506, the prescription history request is validated and provided to a prescription history system, and a prescription history response that includes second data based on the prescription history request is received, over the network from a prescription history system. For instance, the RxHistoryRequest in step 302 may be validated by de-duplicator logic 308, and an associated RxHistoryResponse may be received by augmenter logic 310, via communication interface 302, from a PBM to which the request in step 402 was forwarded or transmitted. The response may include one or more records for transactions/filled prescriptions for the patient. In embodiments, validating may include determining a level of consent, identifying records and/or data elements that are eligible for de-duplication processing, etc., as described herein. In embodiments, a PDMP may be the prescription history system, and in some embodiments a prescription history system may comprise both a PBM and a PDMP where the second data may include data from each of the PBM and the PDMP.

In step 508, the first data is aggregated with the second data in the prescription history response. For example, augmenter 310 of host system 300 may be configured to aggregate data received from the PBM and the pharmacy database. In embodiments, this aggregation may be performed in the context of the response (RxHistoryResponse) in order to provide a complete response to the request.

In step 510, the aggregated data is de-duplicated. De-duplicating data fields as described herein may be performed by de-duplicator logic 308 of host system 300. In embodiments, one or more fields in a record may be de-duplicated, and multiple records in a response may be de-duplicated. Step 510 may comprise one or more sub-steps in embodiments, e.g., step 512 and/or step 514, described below.

In step 512, a record in the prescription history response that is eligible for data de-duplication is identified. For instance, identifier logic 316 and/or matching logic 314 of augmenter logic 310 may be configured to identify data record of the response that are eligible candidates for data de-duplication. Candidate records may include records that are identical, that are substantially identical, and/or the like, as noted in this description. In embodiments, matching logic 314 is configured to determine if records match or substantially match, order to identify duplicated records.

In step 514, the record is removed based on a determination that the record is a duplicated record. For example, removal logic 312 may be configured to remove the identified record from the RxHistoryResponse, as described herein.

In step 516, the prescription history response that excludes the record is provided over the network to the requesting entity. For instance, the prescription history response received in step 506 has its data aggregated with pharmacy database data in step 508, and has duplicate records removed via de-duplicating step 510, resulting in a de-duplicated RxHistoryResponse that no longer includes duplicate records. This de-duplicated RxHistoryResponse is provided to the requesting entity via communication interface 302.

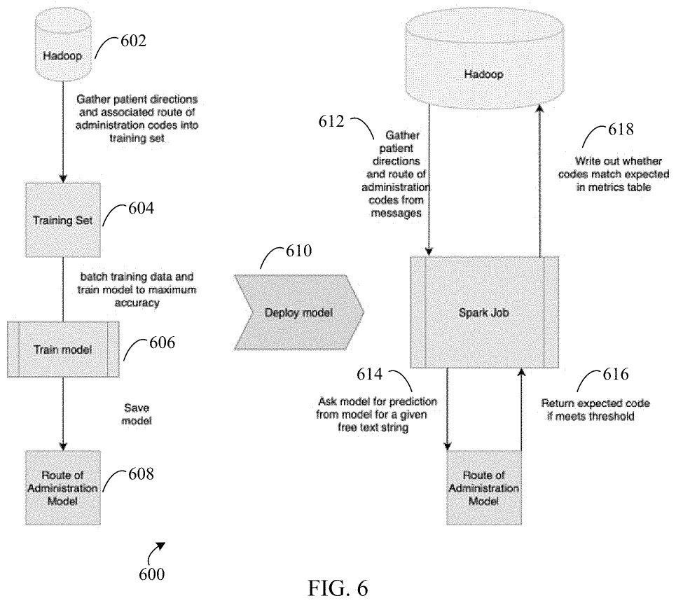

FIGS. 6, 7, and 8 will now be described. As noted herein, neural network models may be trained and implemented in embodiments. In order to codify “sig” free text, embodiments include utilizing a modest sample size of examples of free text with associated codes to train a neural network model that approximates, with a high degree of accuracy, the correct code(s) for a typical free text string. This training data may be sourced from current tables, e.g., as maintained in a Hadoop® cluster, and used to build a training data set by selecting messages that are currently populated with structured sigs. This model is configured to generalize to the problem even if there are some inconsistencies in the data, and the training set is able to be improved as needed over time.

In one example, a model may be trained on 5000 or fewer samples of each code type and takes into account the process for codifying only the route of administration code for classification purposes. In embodiments, a BERT model modified for classification tasks for each code type to be identified is used to classify free text into a route of administration code by adding an output layer specific to the number of possible classes (codes) desired to be classified before training.

An example textual representation of a mode and associated layers is shown below. The model summary below shows the three inputs exemplarily defined, the ‘keras_layer’ includes many layers and encompasses the BERT architecture, and the ‘dense_1’ layer is defined and attached to the BERT layers to classify route of administration.

TABLE 19
Example model summary
Model: “model”

| Layer (type) | Output Shape | Param # | Connected to |
|---|---|---|---|
| input_word_ids | [(None, 128)] | 0 | |
| (InputLayer) | | | |
| input_mask | [(None, 128)] | 0 | |
| (InputLayer) | | | |
| input_type_ids | [(None, 128)] | 0 | |
| (InputLayer) | | | |
| keras_layer | [(None, 768)] | (None, | input_word_ids[0][0] |
| (KerasLayer) | | 109482241) | input_mask[0][0] |
| | | | input_type_ids[0][0] |
| dense_1 (Dense) | (None, 45) | 34605 | keras_layer[0][0] |

Total params: 109,516,846
Trainable params: 109,516,845
Non-trainable params: 1

FIG. 6 shows a flow diagram 600 for data augmentation utilizing a neural network model, in an example embodiment. Flow diagram 600 begins at step 602. In step 602, a number of patient directions are gathered via a Hadoop® cluster with associated route of administration codes for a training data set. In step 604, the batch training data is selected, and in step 606 the batch training data is provided to the BERT model via a neural network. In step 608, the model is saved, and in step 610, the model is subsequently deployed for utilization in the embodiments described herein such as for data augmentation performed according to embodiments via execution of the model described above, e.g., as an Apache Spark™ Software job, or the like.

In embodiments for training, a sig is processed into a tokenized string as shown below, and every token is turned into a representative integer. There are also special tokens added to inform the model about the inputs.

TABLE 20
Tokenized string for model training
Tokenized free text input:

| | | | | | | | | | | | | | |
|---|---|---|---|---|---|---|---|---|---|---|---|---|---|
| [101 | 6611 | 1015 | 4646 | 2000 | 1996 | 3096 | 1016 | 2335 | 3679 | 2005 | 2403 | 2420 | 1012 |
| 102 | 0 | 0 | 0 | 0 | 0 | 0 | 0 | 0 | 0 | 0 | 0 | 0 | 0 |
| 0 | 0 | 0 | 0 | 0 | 0 | 0 | 0 | 0 | 0 | 0 | 0 | 0 | 0 |
| 0 | 0 | 0 | 0 | 0 | 0 | 0 | 0 | 0 | 0 | 0 | 0 | 0 | 0 |
| 0 | 0 | 0 | 0 | 0 | 0 | 0 | 0 | 0 | 0 | 0 | 0 | 0 | 0 |
| 0 | 0 | 0 | 0 | 0 | 0 | 0 | 0 | 0 | 0 | 0 | 0 | 0 | 0 |
| 0 | 0 | 0 | 0 | 0 | 0 | 0 | 0 | 0 | 0 | 0 | 0 | 0 | 0 |
| 0 | 0 | 0 | 0 | 0 | 0 | 0 | 0 | 0 | 0 | 0 | 0 | 0 | 0 |
| 0 | 0 | 0 | 0 | 0 | 0 | 0 | 0 | 0 | 0 | 0 | 0 | 0 | 0 |
| 0 | 0] | | | | | | | | | | | | |

String view: [‘apply’, ‘1’, ‘application’, ‘to’, ‘the’, ‘skin’, ‘2’, ‘times’, ‘daily’, ‘for’, ‘14’, ‘days’, ‘.’]
Code id: 8 Code: 359540000 Text: Topical

In embodiments, training includes running batches of these samples through the model and updating the model to gradually produce better predictions. The logic that updates model may be handled by an open source deep learning library, in embodiments.

In step 612, patient direction and route of administration codes may be gathered from the Hadoop® cluster, based on messages or requests as noted herein, for the model execution. The executing model is provided with the gathered data to determine a prediction for a free text string, in step 614. The model determines an expected code in step 616, and if the expected code confidence score meets or exceeds a confidence threshold, the expected code is used, via data augmentation, to replace the free text field on which the code is based. In step 618, post-prediction data is gathered to determine and/or improve the model accuracy. In embodiments, model accuracy may be subsequently checked based on a validation set that was not used during model training and/or on data collected during implementation of the model. A model for predicting the actual route of administration code has been shown to predict with at least 99.62% accuracy.

The example model described makes predictions by taking an input string and producing a set of probabilities for each code it knows about. The largest value in the set can be considered the prediction, in embodiments. Additionally, a confidence threshold may be chosen/implemented to determine whether a prediction should be provided at all.

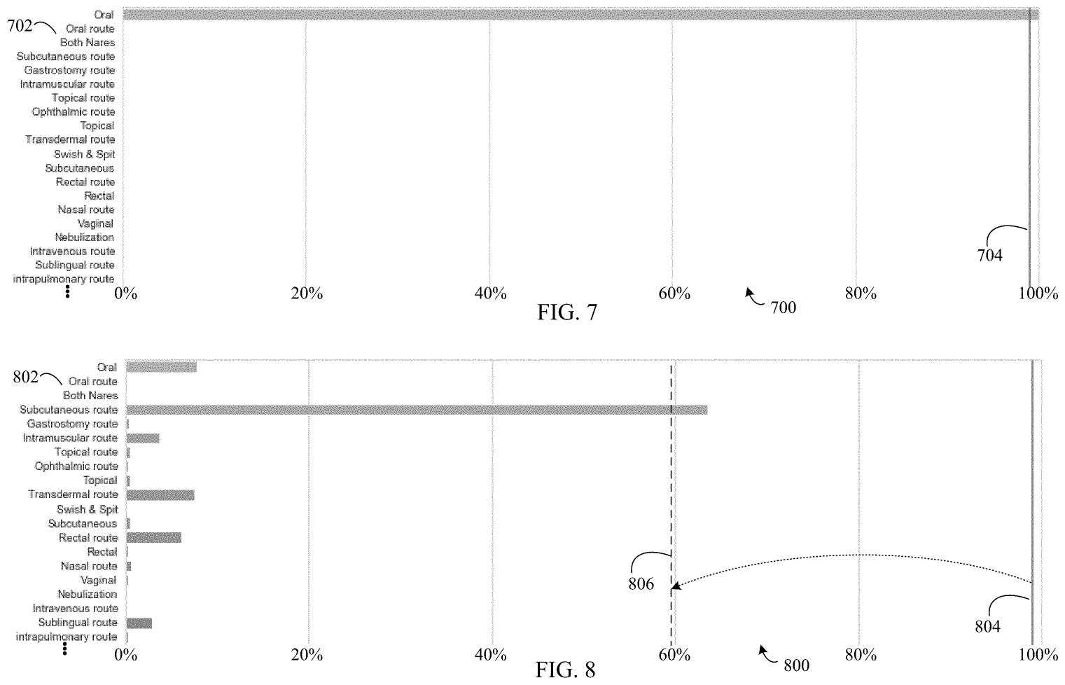

FIGS. 7 and 8 are now described in the context of the example model and FIG. 6. The below predictions are plotted as bar charts with each bar representing the confidence score for that code (not all possible codes are shown for brevity and illustrative clarity). FIGS. 7 and 8 include an example threshold line at the 0.99 score (i.e., 99%) that may be used, e.g., in embodiments to only make predictions with very high confidence.

FIG. 7 shows a graphical representation of a code classification 700 generated by a neural network model, in an example embodiment. Code classification 700 includes an example list of codes 702 on the y-axis and a percent confidence score (%) on the x-axis. The confidence threshold 704 as described above is also shown. In this example, the model is tasked to identify the free text sig “take 1 capsule by mouth daily” to be a codified sig code, i.e., an “oral” route based on the free text “by mouth.” As “by mouth” may be a common free text sig provided, the model determines the code as an “oral” route with 100% confidence, in which case, this code may be returned and used for data augmentation embodiments herein.

Similarly, a free text sig of “orally” may result in the same prediction for an “oral” code. However, some free text sigs may be uncommon and more difficult to classify and result in uncertainty, as noted below.

FIG. 8 shows a graphical representation of a code classification 800 generated by a neural network model, in an example embodiment. Code classification 800 includes an example list of codes 802 on the y-axis and a percent confidence score (%) on the x-axis. The confidence threshold 804 as described above is also shown. In this example, the model is tasked to identify the free text sig “take 1 capsule under the arm daily” to be a codified sig code. With an unorthodox sig such as this one, the model may not provide a resulting code with confidence near the threshold. For instance, as shown, a number of possible codes have received a score in this example, with a “subcutaneous” route receiving the highest score of approximately 65%, well below confidence threshold 804 set at 99%. According to embodiments, a code may not be returned for use in data augmentation embodiments herein.

In alternate embodiments, as noted above, the confidence threshold may be dynamically adjusted based on the data field, the free text sig, the code, etc. Here, this dynamic adjustment is shown as adjusted threshold 806 which is set to approximately 60%. In such a case, the code for “subcutaneous” route may be returned for use in data augmentation embodiments herein.

### III. Further Example Embodiments and Advantages

In some example embodiments, one or more of the operations of the flowcharts described herein may not be performed. Moreover, operations in addition to or in lieu of the operations of the flowcharts described herein may be performed. Further, in some example embodiments, one or more of the operations of the flowcharts described herein may be performed out of order, in an alternate sequence, or partially (or completely) concurrently with each other or with other operations.

Embodiments and techniques, including methods, described herein may be performed in various ways such as, but not limited to, being implemented by hardware, or hardware combined with one or both of software and firmware.

In embodiments, data de-duplication may be performed as part of validating a RxHistoryResponse and/or as part of aggregating data from pharmacy fill records and/or PDMP data with PBM paid claim data in the Response, and may include one or more of: identifying data elements that are eligible for de-duplication processing; executing de-duplication logic on records or Response data; and/or removing any duplicated dispensed medication records from the aggregation.

In embodiments, data augmentation may be performed as part of aggregating data from pharmacy fill records or PDMP data with PBM paid claim data in the Response, and may include one or more of: identifying data elements that are eligible for augmentation processing and have not already been populated by the data suppliers (pharmacy/PBM/PDMP); augmenting the data elements found in each unique medication dispensed occurrence using the appropriate identified data source, including one or more of a host directory services or a drug compendia; and/or tracking each medication dispensed occurrence that has been augmented with augmenting data.

As described herein, embodiments utilize de-duplication and augmentation techniques, including neural network models, to effectively and efficiently remove duplicated records while also completing and codifying such records which decreases memory footprint via de-duplication, and also reduces network utilization via de-duplication and augmentation (e.g., by providing complete and correct responses to requests that do not require additional network transactions)—this allows for the processing of requests to be accomplished more efficiently. Embodiments also allow for the tracking of augmented data that may later be used to further improve neural network models and provide more robust data integrity. That is, the embodiments herein utilize a unique combination of de-duplication and augmentation for data that provide for improved data accuracy and resource efficiencies that was previously not available for software services, much less for the specific embodiments described herein.

Embodiments herein also provide for receiving data from PDMPs that is subject to de-duplication and/or augmentation. When a pharmacy fills a controlled substance such as an opioid, they may be mandated to send a record of the dispensed drug to a state entity, called a PMP or a PDMP, so that the state has record. States may mandate that prescribers and pharmacists check PDMP databases for records to ensure patients are not getting too many opioids or are not redirecting opioids. If an opioid prescription was filled at participating pharmacy, or claimed through participating PBM, it may be a duplicate. The same or similar fields may be used to confirm duplicates and eligible fields for augmentation as outlined in the described embodiments herein.

### IV. Example Processing Device Implementations

Data de-duplication and data augmentation system and device embodiments described herein, such as systems of FIG. 1, systems of FIG. 2, host system 300 of FIG. 3, along with any respective components/subcomponents and/or further embodiments thereof, and/or any flowcharts, execution flows, further systems, sub-systems, and/or components, including other network-connected devices, disclosed herein may be implemented in hardware (e.g., hardware logic/electrical circuitry), or any combination of hardware with one or both of software (computer program code or instructions configured to be executed in one or more processors or processing devices) and firmware. In embodiments with respect to the example computer implementations in this Section, main memory, memory cards and memory sticks, memory devices, and/or the like may include and or implement the described techniques and embodiments.

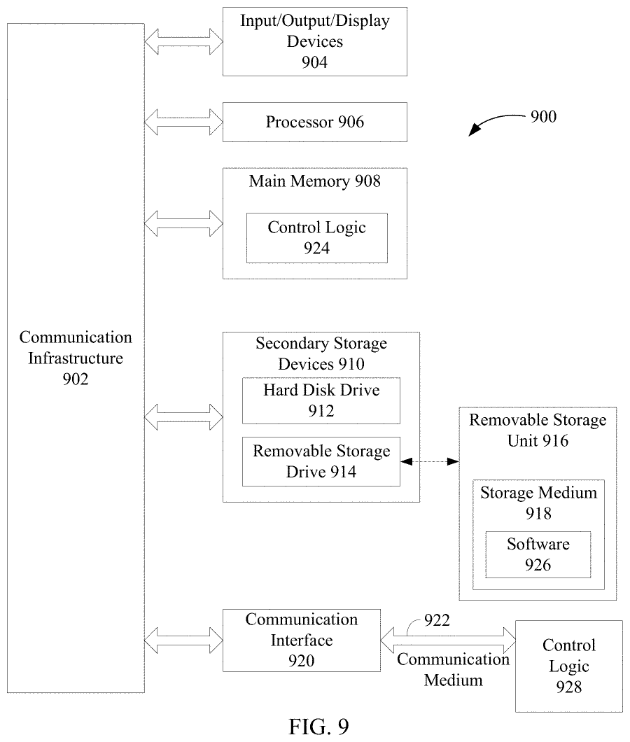

The embodiments described herein, including devices, systems, methods/processes, and/or apparatuses, may be implemented in or using processing devices, communication systems, servers, and/or, computers, such as a processing device 900 shown in FIG. 9. It should be noted that processing device 900 may represent mobile devices, communication devices/systems, entertainment systems/devices, processing devices, and/or traditional computers in one or more embodiments. For example, a resource generation system as described herein, and any of the sub-systems and/or components respectively contained therein and/or associated therewith, along with further embodiments thereof, may be implemented in or using one or more processing devices 900 and/or similar computing devices.

Processing device 900 can be any commercially available and well known communication device, processing device, and/or computer capable of performing the functions described herein, such as devices/computers available from International Business Machines®, Apple®, Sun®, HP®, Dell®, Cray®, Samsung®, Nokia®, etc. Processing device 900 may be any type of computer, including a desktop computer, a server, etc., and may be a computing device or system within another device or system.

Processing device 900 includes one or more processors (also called central processing units, or CPUs), such as a processor 906. Processor 906 is connected to a communication infrastructure 902, such as a communication bus. In some embodiments, processor 906 can simultaneously operate multiple computing threads, and in some embodiments, processor 906 may comprise one or more processors.

Processing device 900 also includes a primary or main memory 908, such as random access memory (RAM). Main memory 908 has stored therein control logic 924 (computer software), and data.

Processing device 900 also includes one or more secondary storage devices 910. Secondary storage devices 910 include, for example, a hard disk drive 912 and/or a removable storage device or drive 914, as well as other types of storage devices, such as memory cards and memory sticks. For instance, processing device 900 may include an industry standard interface, such a universal serial bus (USB) interface for interfacing with devices such as a memory stick. Removable storage drive 914 represents a floppy disk drive, a magnetic tape drive, a compact disk drive, an optical storage device, tape backup, etc.

Removable storage drive 914 interacts with a removable storage unit 916. Removable storage unit 916 includes a computer useable or readable storage medium 918 having stored therein computer software 926 (control logic) and/or data. Removable storage unit 916 represents a floppy disk, magnetic tape, compact disk, DVD, optical storage disk, or any other computer data storage device. Removable storage drive 914 reads from and/or writes to removable storage unit 916 in a well-known manner.

Processing device 900 also includes input/output/display devices 904, such as touchscreens, LED and LCD displays, monitors, keyboards, pointing devices, etc.

Processing device 900 further includes a communication or network interface 920. Communication interface 920 enables processing device 900 to communicate with remote devices. For example, communication interface 920 allows processing device 900 to communicate over communication networks or mediums 922 (representing a form of a computer useable or readable medium), such as LANs, WANs, the Internet, etc. Network interface 920 may interface with remote sites or networks via wired or wireless connections.

Control logic 928 may be transmitted to and from processing device 900 via the communication medium 922.

Any apparatus or manufacture comprising a computer useable or readable medium having control logic (software) stored therein is referred to herein as a computer program product or program storage device. This includes, but is not limited to, processing device 900, main memory 908, secondary storage devices 910, and removable storage unit 916. Such computer program products, having control logic stored therein that, when executed by one or more data processing devices, cause such data processing devices to operate as described herein, represent embodiments.

Techniques, including methods, and embodiments described herein may be implemented by hardware (digital and/or analog) or a combination of hardware with one or both of software and/or firmware. Techniques described herein may be implemented by one or more components. Embodiments may comprise computer program products comprising logic (e.g., in the form of program code or software as well as firmware) stored on any computer useable medium, which may be integrated in or separate from other components. Such program code, when executed by one or more processor circuits, causes a device to operate as described herein. Devices in which embodiments may be implemented may include storage, such as storage drives, memory devices, and further types of physical hardware computer-readable storage media. Examples of such computer-readable storage media include, a hard disk, a removable magnetic disk, a removable optical disk, flash memory cards, digital video disks, random access memories (RAMs), read only memories (ROM), and other types of physical hardware storage media. In greater detail, examples of such computer-readable storage media include, but are not limited to, a hard disk associated with a hard disk drive, a removable magnetic disk, a removable optical disk (e.g., CDROMs, DVDs, etc.), zip disks, tapes, magnetic storage devices, MEMS (micro-electromechanical systems) storage, nanotechnology-based storage devices, flash memory cards, digital video discs, RAM devices, ROM devices, and further types of physical hardware storage media. Such computer-readable storage media may, for example, store computer program logic, e.g., program modules, comprising computer executable instructions that, when executed by one or more processor circuits, provide and/or maintain one or more aspects of functionality described herein with reference to the figures, as well as any and all components, capabilities, and functions therein and/or further embodiments described herein.

Such computer-readable storage media are distinguished from and non-overlapping with communication media and modulated data signals (i.e., do not include communication media or modulated data signals). Communication media embodies computer-readable instructions, data structures, program modules or other data in a modulated data signal such as a carrier wave. The term “modulated data signal” means a signal that has one or more of its characteristics set or changed in such a manner as to encode information in the signal. By way of example, and not limitation, communication media includes wireless media such as acoustic, RF, infrared and other wireless media, as well as wired media and signals transmitted over wired media. Embodiments are also directed to such communication media.

The techniques and embodiments described herein may be implemented as, or in, various types of circuits, devices, apparatuses, and systems. For instance, embodiments may be included, without limitation, in processing devices (e.g., illustrated in FIG. 9) such as computers and servers, as well as communication systems such as switches, routers, gateways, and/or the like, communication devices such as smart phones, home electronics, gaming consoles, entertainment devices/systems, etc. A device, as defined herein, is a machine or manufacture as defined by 35 U.S.C. § 101. That is, as used herein, the term “device” refers to a machine or other tangible, manufactured object and excludes software and signals. Devices may include digital circuits, analog circuits, or a combination thereof. Devices may include one or more processor circuits (e.g., central processing units (CPUs), processor 906 of FIG. 9), microprocessors, digital signal processors (DSPs), and further types of physical hardware processor circuits) and/or may be implemented with any semiconductor technology in a semiconductor material, including one or more of a Bipolar Junction Transistor (BJT), a heterojunction bipolar transistor (HBT), a metal oxide field effect transistor (MOSFET) device, a metal semiconductor field effect transistor (MESFET) or other transconductor or transistor technology device. Such devices may use the same or alternative configurations other than the configuration illustrated in embodiments presented herein.

### V. Conclusion

While various embodiments have been described above, it should be understood that they have been presented by way of example only, and not limitation. It will be apparent to persons skilled in the relevant art that various changes in form and detail can be made therein without departing from the spirit and scope of the embodiments. Thus, the breadth and scope of the embodiments should not be limited by any of the above-described exemplary embodiments, but should be defined only in accordance with the following claims and their equivalents.

What is claimed is:

1. A method for data augmentation performed by a computing device, the method comprising:
receiving, over a network from a requesting entity, a prescription history request for a patient for whom prescriptions were provided;
acquiring first data from a pharmacy database based on the prescription history request;
receiving, over the network from a prescription history system, a prescription history response that includes second data based on the prescription history request;
aggregating the first data with the second data in the prescription history response;
augmenting a data field with the augmenting data by:
identifying the data field in the prescription history response that is eligible for data augmentation; and
retrieving augmenting data from a determined data source based at least on the data field and an associated data field in the prescription history response; and
providing, over the network to the requesting entity, the prescription history response that includes the augmenting data.

2. The method of claim 1, wherein identifying the data field in the prescription history response that is eligible for data augmentation includes identifying the data field as being a blank data field; and
wherein the determined data source is one or more of a drug compendia or a host directory.

3. The method of claim 1, wherein identifying the data field in the prescription history response that is eligible for data augmentation includes identifying the data field as including the free text sig information; and
wherein retrieving augmenting data from the determined data source includes determining a structured code that corresponds to free text sig information in the data field by utilizing a neural network model that generalizes the free text sig information according to referential matching training of free text and structured code types.

4. The method of claim 3, wherein the free text sig information is classified to a code by the neural network model according to one of a plurality of output layers thereof that is specifically associated with a structured code type.

5. The method of claim 3, wherein said determining the structured code includes meeting or exceeding a confidence threshold for the structured code by the neural network model, the confidence threshold being dynamically configurable based on at least one of the structured code or a type of the structured code.

6. The method of claim 1, wherein identifying the data field in the prescription history response that is eligible for data augmentation includes identifying the data field as being a populated field having pre-populated data; and
wherein the determined data source is one or more of a drug compendia or a host directory;
the method further comprising:
determining that the data in the data field does not conform with an existing standard format; and
modifying the pre-populated data with the augmenting data that corresponds to thereto.

7. The method of claim 1, further comprising:
generating tracking information related to the augmenting data; and
providing the tracking information with the prescription history response.

8. The method of claim 1, wherein the computing device comprises a cloud-based host device; and
wherein said augmenting the data field with the augmenting data is performed in real-time subsequent to said acquiring the first data and said receiving, over the network from the prescription history system, the prescription history response.

9. A computer-readable storage medium having program instructions encoded thereof that, when executed by one or more processors, performs a method for data augmentation, the method comprising:
receiving, over a network from a requesting entity, a prescription history request for a patient for whom prescriptions were provided;
acquiring first data from a pharmacy database based on the prescription history request;
receiving, over the network from a prescription history system, a prescription history response that includes second data based on the prescription history request;
aggregating the first data with the second data in the prescription history response;
augmenting a data field with the augmenting data by:
identifying the data field in the prescription history response that is eligible for data augmentation; and
retrieving augmenting data from a determined data source based at least on the data field and an associated data field in the prescription history response; and
providing, over the network to the requesting entity, the prescription history response that includes the augmenting data.

10. The computer-readable storage medium of claim 9, wherein identifying the data field in the prescription history response that is eligible for data augmentation includes identifying the data field as being a blank data field; and
wherein the determined data source is one or more of a drug compendia or a host directory.

11. The method of claim 9, wherein identifying the data field in the prescription history response that is eligible for data augmentation includes identifying the data field as including the free text sig information; and
wherein retrieving augmenting data from the determined data source includes determining a structured code that corresponds to free text sig information in the data field by utilizing a neural network model that generalizes the free text sig information according to referential matching training of free text and structured code types.

12. The computer-readable storage medium of claim 11, wherein the free text sig information is classified to a code by the neural network model according to one of a plurality of output layers thereof that is specifically associated with a structured code type.

13. The computer-readable storage medium of claim 11, wherein said determining the structured code includes meeting or exceeding a confidence threshold for the structured code by the neural network model, the confidence threshold being dynamically configurable based on at least one of the structured code or a type of the structured code.

14. The computer-readable storage medium of claim 9, wherein identifying the data field in the prescription history response that is eligible for data augmentation includes identifying the data field as being a populated field having pre-populated data;
wherein the determined data source is one or more of a drug compendia or a host directory; and
wherein the method further comprises:
determining that the data in the data field does not conform with an existing standard format; and
modifying the pre-populated data with the augmenting data that corresponds to thereto.

15. The computer-readable storage medium of claim 9, wherein the method further comprises:
determining that no augmenting data is available for an identified data field eligible for data augmentation; and
providing with the prescription history response at least one of:
a data element name;
an expected data source; or
a description of one or more reasons for data unavailability.

16. The computer-readable storage medium of claim 9, wherein said augmenting the data field with the augmenting data includes augmenting a plurality of data fields with a respective plurality of augmenting data; and
wherein the method further comprises determining that each of the plurality of data fields is associated with a unique medication dispensed occurrence in the prescription history response.

17. A system comprising:
at least one processor; and
at least one memory device storing program instructions that, when executed by the at least one processor, perform a method, the method comprising:
receiving, over a network from a requesting entity, a prescription history request for a patient for whom prescriptions were provided;
acquiring first data from a pharmacy database based on the prescription history request;
receiving, over the network from a prescription history system, a prescription history response that includes second data based on the prescription history request;
aggregating the first data with the second data in the prescription history response;
augmenting a data field with the augmenting data by:
identifying the data field in the prescription history response that is eligible for data augmentation;
retrieving reference data from a determined data source based at least on the data field and an associated data field in the prescription history response; and
calculating a numerical value, as augmenting data, based at least on the data field, the reference data, and an associated data field in the prescription history response; and
providing, over the network to the requesting entity, the prescription history response that includes the augmenting data.

18. The system of claim 17, wherein the numerical value is at least one of a dosage, a dosage unit, an opioid risk score, a number of refills remaining, or cancelation date.

19. The system of claim 17, wherein the method comprises:
augmenting an additional data field with additional augmenting data by:
identifying an additional data field in the prescription history response that is eligible for data augmentation; and
retrieving additional augmenting data from at least one of the determined data source or an additionally determined data source based at least on the additional data field and an additional associated data field in the prescription history response;
wherein the prescription history response that includes the augmenting data also includes the additional augmenting data.

20. The system of claim 17, wherein the method further comprises:
generating tracking information related to the augmenting data; and
providing the tracking information with the prescription history response.
